(19) United States
(12) Patent Application Publication
(10) Pub. No.: US 2014/0100533 A1
(43) Pub. Date: Apr. 10, 2014

(54) Protective Intravenous Line End Cap

(71) Applicant: Peter Lyons, Greendale, WI (US)

(72) Inventor: Peter Lyons, Greendale, WI (US)

(21) Appl. No.: 13/647,697

(22) Filed: Oct. 9, 2012

(51) Int. Cl. A61M 39/20

(52) U.S. Cl. 604/264

(57) ABSTRACT

An annular cap for covering a lumen including first and second elongate members each having inside and outside surfaces, proximal and distal end portions and two opposite longitudinal edges. The cap having an interlocking snap member positioned between and connected to the first and second elongate members along the opposite longitudinal edges, the first and second elongate members being pivotable with respect to each other about a pivot axis. The elongate members interlock over and around the lumen to provide complete enclosure of the lumen when the cap is in a closed position.

### FIELD

This invention relates to intravenous medication administration and more specifically to a cap which interlocks over and around an intravenous line end to provide complete enclosure of the intravenous line end.

### BACKGROUND

Intravenous (“IV”) therapy is a useful and effective ways to administer medication and medical fluids to patients. IV medication administration is the infusion of liquid substances directly into a vein in the body. Intravenous therapy is traditionally used to correct electrolyte imbalances, to deliver medications, for blood transfusion or as fluid replacement to correct, for example, dehydration.

Direct infusion into the body's circulatory system has the benefit of producing a rapid response and result. This is advantageous for situations in which quick treatment of the patient is needed. However, once medication or fluid is administered, it cannot be withdrawn from the circulatory system or its action terminated. Thus, extreme caution is necessary for patient safety prior to the administration of medication or other liquid, this includes correct identification of IV lines.

IV lines of the prior art exist in many forms and are generally categorized as central or peripheral lines. Central Lines have lumens which are the portion of the IV line that emerges from the patient's skin The prior art consists of single, double, triple or even quadruple lumens. With a central line, multiple lumens enter one point through the skin (such a configuration appears to be one line but actually consists of individual lines going through a larger tube). Each lumen has its own delivery site or exit site inside the blood vessel. The end portion of a lumen is typically referred to as “needle free valve” or “IV valve.” Valves come in a variety of shapes and sizes however, all use a “lure lock” system or standardized IV-line connection system.

A peripheral line IV includes a cannula. A cannula enters the patient's vein and includes a portion called a “hub” which emerges from the patient's skin IV line extension tubing, otherwise known as a lumen, attaches to the hub. Attached to the end of the IV line extension is a “needle free valve” or “IV valve.” These valves also come in a variety of sizes and typically include the “lure lock” system. This system is a method of connecting and disconnecting IV lines and standardizes IV sets.

As noted above, an IV line may have a singular access point into a vein but it may also have multiple lines from a single access point called lumens. Multiple access points can present multiple problems. Traditionally, IV lines can have multiple yet specific purposes. Some IV lines are designated for singular use such as, for example, Total Parenteral Nutrition. In some situations, once a particular lumen is used for a designated medication, no other medication should be used in that lumen. Therefore, maintaining a particular lumen for a particular purpose is critical.

Careless handling of medications can result in medication not being administered, being administered incorrectly or being administered through a lumen which is not designated for a particular medication or purpose. For example, certain medications may make a lumen or a line prone to claudication (such as antibiotics). When a line is occluded certain medications can be inserted into a the lumen but they must remain in the line for up to an hour before removal of the occlusion and the medication. Such a practice can be timely and avoidable.

Furthermore, current communication by medical personnel is by paper and computerized charting. There is no effective direct method of labeling lumens. It is typical for multiple staff members, including primary staff nurses, an IV nurse, lab draw personal and staff physicians to each use a patient's lumens throughout the day. If medical personnel do not take the time to go through a patient's (sometimes lengthy) medical chart personnel may be unaware of important information related to medications and/or fluids which have been administered. Even when medical personnel do refer to a patient's medical chart sometimes documentation has not been timely charted or fully completed. All of these potential problems can have a significant impact on a patient's well-being, ability to recovery and the time it takes to do so.

Current methods of labeling multiple lumens include using colored IV lines, colored tape or screw-on type enclosures. These methods have numerous shortcomings. For example, if an existing IV line has a designated color, the IV line cannot be changed without removal and reinsertion of a new line which increases the chance of infection in the patient. Such a practice is also timely and can cause the patient unnecessary discomfort.

Colored tape can also be used on an IV line to designate a particular purpose for a lumen. Tape however, cannot generally be placed directly onto the lumen because of space restrictions. Using tape to label lumens also has shortcomings besides space restrictions. Tape, for example, can leave an adhesive residue which can house bacteria. Tape is also not effective because it can be hard to read and can quickly loose its adhesive ability. Furthermore, tape cannot be attached to the end of the lumen or used to block the end of the lumen to prevent improper medication administration. Therefore, tape must be secured to the IV line connected to the lumen. This causes medical personal to have to trace the line back to the colored tape on the lumen at the end of the IV line. This can lead to error with multiple lumens and can also be time consuming in emergency situations.

Screw-on type enclosures which cover a small, distal most portion of a lumen are also used. These enclosures can be time consuming and difficult to use as they require two hands to operate/secure and once secured only cover a small section of a lumen. These enclosures also only cover the lumen end for sanitary purposes and do not serve a identification function. It would be advantageous to have a cap which is small and slim so that it can be easily maneuvered by medical personnel.

It would be desirable to have a lumen cap which overcomes these problems by interlocking completely over and around a lumen to provide complete enclosure of the lumen when the cap is in a closed position. It would also be desirable to have a lumen cap which is color-coded to designate particular medications/fluids which are safe to administer through a particular IV line. This would help to prevent possible medication/fluid administration errors by medical personnel.

Furthermore, it would be desirable to have a lumen cap which is able to be labeled by a writing utensil so as to save medical personnel valuable time by reducing the need to extensively read through medical charts. It would be desirable to have a lumen cap which is easy and quick to place on a lumen. Finally, it would be desirable to have a lumen cap which is durable as well as easy and inexpensive to manufacture.

This device meets these needs and overcomes other problems and shortcomings in the prior art.

### SUMMARY

The device is a protective cap for covering a lumen for use primarily in healthcare settings such as hospitals and nursing home facilities. In its most preferred form, the cap is configured to be placed around a lumen so that the entire lumen is covered and protected when the cap is in the closed position.

The device includes first and second elongate members each including inside and outside surfaces, proximal and distal end portions and two opposite longitudinal edges. It is most preferred that an interlocking snap member is positioned between and connected to the first and second elongate members along the opposite longitudinal edges, the first and second elongate members being pivotable with respect to each other about a pivot axis. It is highly preferred that the elongate members interlock over and around the lumen to provide complete enclosure of the lumen when the cap is in a closed position.

During use, an open cap is placed around a lumen and the cap is then moved to the closed position thereby completely enclosing the entire lumen. During closure, the first and second elongate members pivot towards one another along an axis until the interlocking snap member is engaged and the cap secured.

It is highly preferred that the proximal and distal ends each include an annular opening for passage of an intravenous line there through, the intravenous line being connected to the lumen. Typically, when the cap is secured to the lumen, an IV line will only pass through the annular opening either in the proximal end or the distal end. By including an annular opening on both the proximal and distal ends the cap is able to be used in either direction. Preferably, the annular opening on the proximal end is identical to the annular opening on the distal end and is at least 2.0 mm or greater in diameter.

It is preferred that the inside surfaces frictionally engage the lumen and prevent removal of the lumen when in the closed position. Additionally, the second elongate member includes at least one grip flange for opening and closing of the cap and that each grip flange be integrally formed with the second elongate member.

Preferably at least one hinge connects the two opposite longitudinal edges although more than one hinge and a variety of hinge types can be used. It is preferable that the cap also include a label-receiving member on the outside surface of the first elongate member and/or second elongate member for designation of patient care instructions. The label-receiving member would be sized to include messages such as “TPN Only,” “Do Not Use,” “Lab Draw,” or a pager/phone number for medical personnel. It is highly preferable that the cap be color-coded to indicate corresponding medications/fluids or patient care instructions set by hospital policy.

Another highly preferred embodiment of the device is a cap for covering a portion of an intravenous line. Preferably the cap includes first and second elongate members each including inside and outside surfaces, proximal and distal end portions and two opposite longitudinal edges; and an interlocking snap member positioned between and connected to the first and second elongate members along the opposite longitudinal edges, the first and second elongate members being pivotable with respect to each other about a pivot axis. The snap member interlocks over and around an intravenous line to provide complete enclosure when the cap is in a closed position.

It is highly preferred that the proximal and distal ends each include an annular opening for passage of the intravenous line there through and that each annular opening is sized to allow movement of the cap along a length of the intravenous line.

An alternate use for the cap is at the pump end of the IV system. At the pump end is an “IV tubing set.” The set has a line that enters the IV bag which then travels to an IV pump or a drip chamber to regulate the flow of fluid into the patient. From the pump or drip chamber the line is then connected to the lumen with a lure lock connector. The diameter/size of the IV line is the same as the diameter/size of the line on the lumen, and additionally, the line on the delivery end also uses the lure lock connecting end. Therefore, the cap will also fit the delivery end of the line and can be used to cover the delivery end (adjacent an IV bag). This is a secondary use for the cap. The cap on the delivery end of an IV line would preferably be color-coded and/or include a label-receiving member to designate particular medications/fluids which are safe to administer through a particular IV line.

In descriptions of this invention, including in the claims below, the terms “comprising,” “including” and “having” (each in their various forms) and the term “with” are each to be understood as opened-ended, rather than limiting, terms.

### BRIEF DESCRIPTION OF THE DRAWINGS

The drawings illustrate a preferred embodiment including the above-noted characteristics and features of the device. The device will be readily understood from the descriptions and drawings. In the drawings: FIG. 1 is a perspective view of an embodiment of the inventive cap.

FIG. 2 is a top view of the cap of FIG. 1 illustrating the lumen in place.

FIG. 3 a perspective view of the cap of FIG. 1 with the lumen removed from the cap.

FIG. 4 is a side view of the cap of FIG. 1 in the open position.

FIG. 5 is a top view of the cap of FIG. 1.

FIG. 6 is a side view of the cap of FIG. 1 in the closed position.

FIG. 7 is a perspective view of the cap of FIG. 1.

FIG. 8 is a perspective view of the cap of FIG. 1 illustrating an IV line within the cap in the open position.

FIG. 9 is a perspective view of the cap of FIG. 1 engaged with an IV line in the closed position.

### DETAILED DESCRIPTION

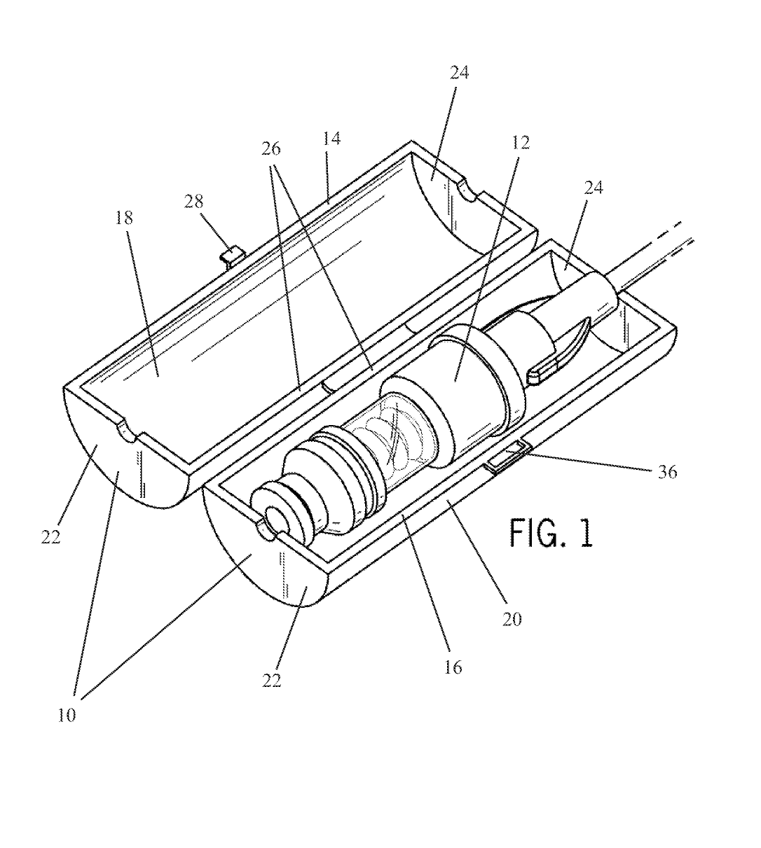

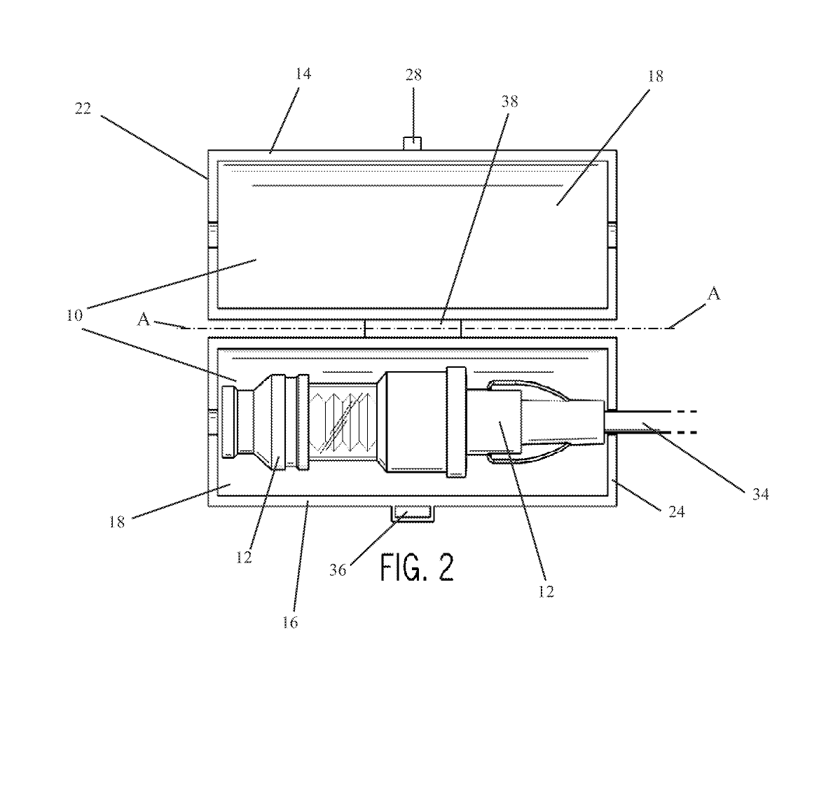

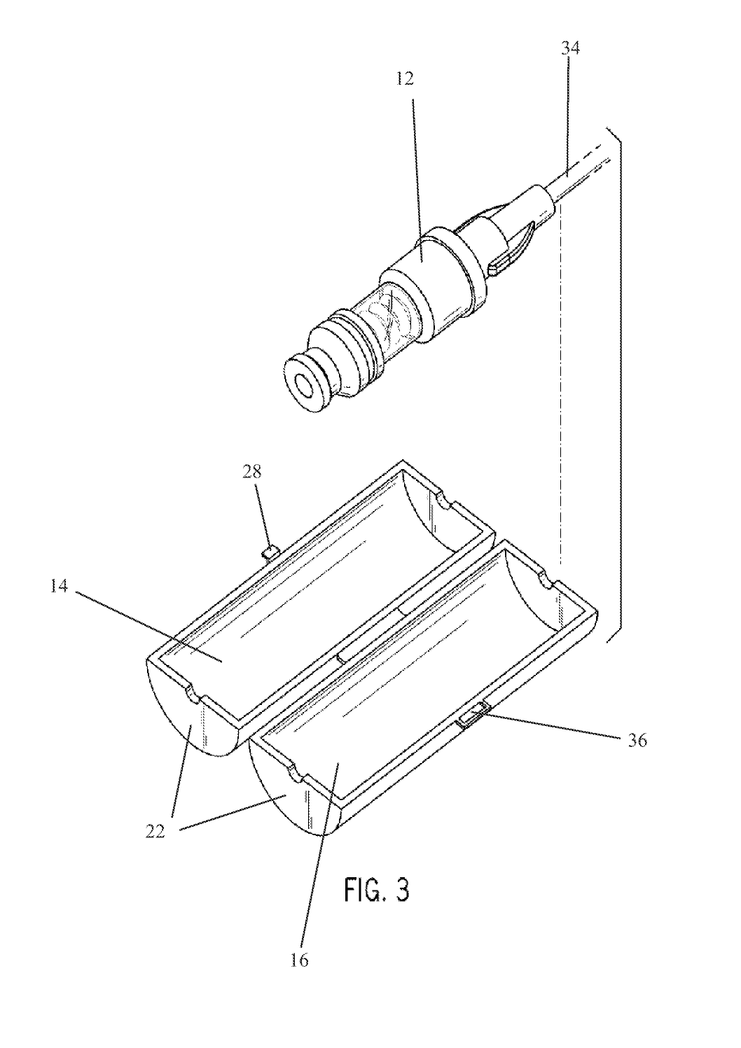

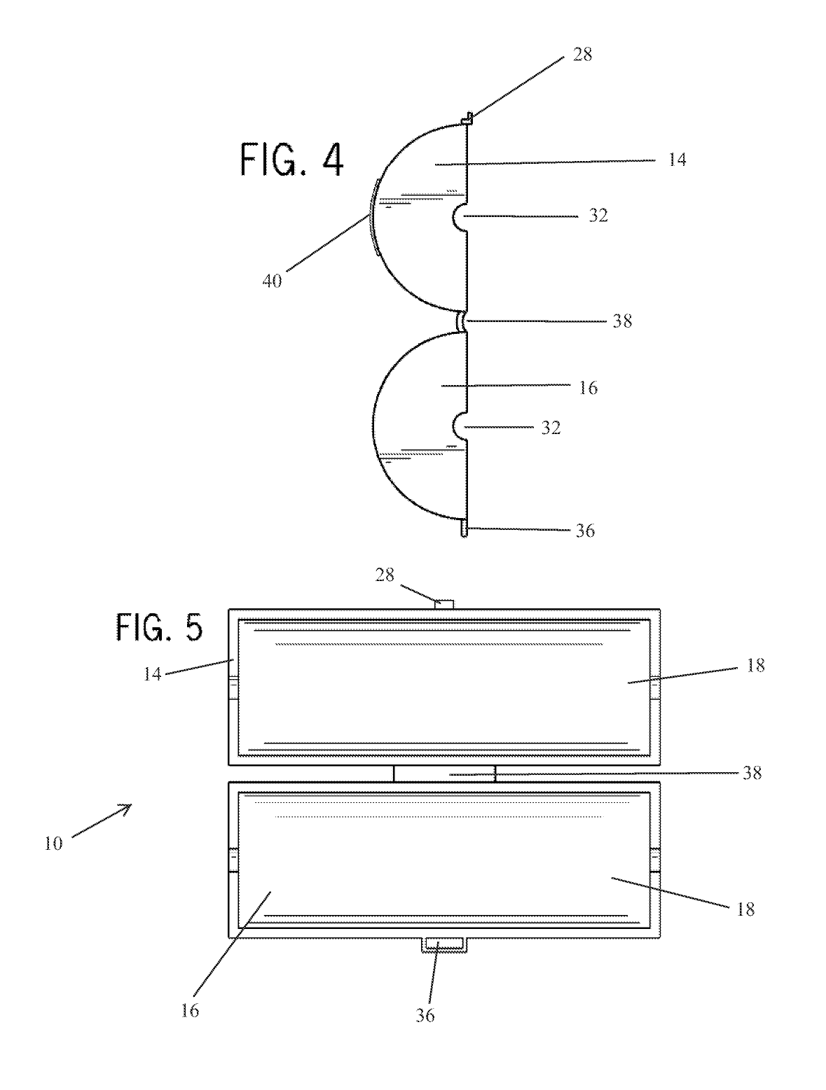

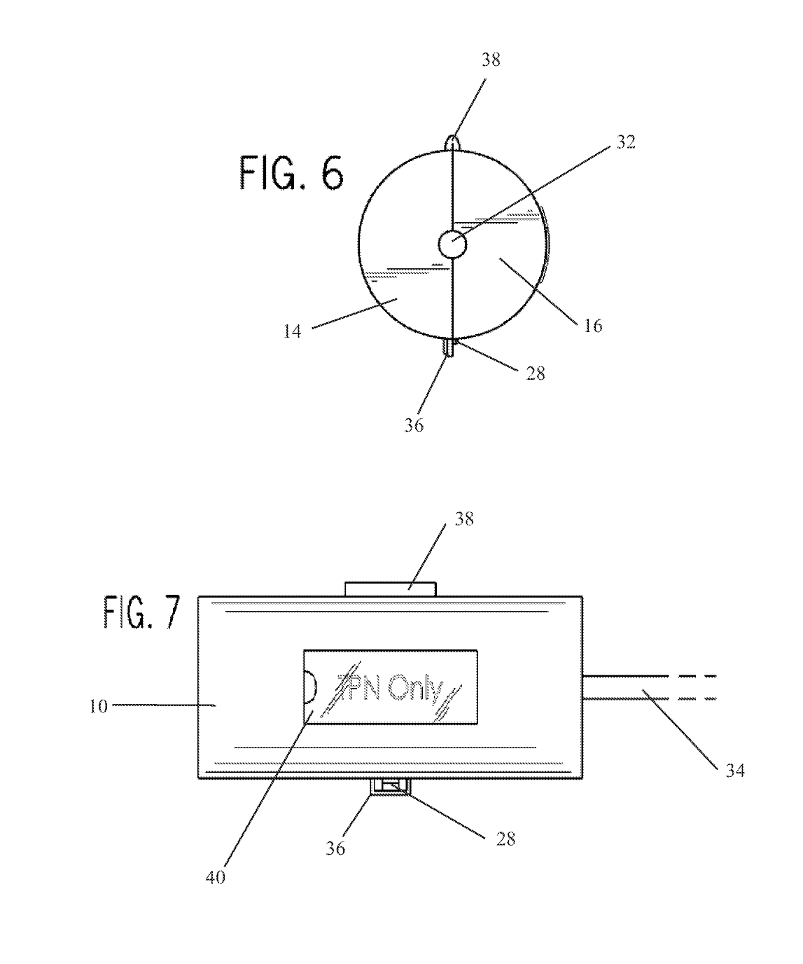

As shown in FIGS. 1-9, the device is an annular cap 10 for covering a lumen 12. Cap 10 as illustrated in the drawings includes first and second elongate members 14, 16 each having inside and outside surfaces 18, 20, proximal and distal end portions 22, 24 and two opposite longitudinal edges 26. Interlocking snap member 28 is positioned between and connected to first and second elongate members 14, 16 along opposite longitudinal edges 26. First and second elongate members 14, 16 are pivotable with respect to one other about a pivot axis A which extends along their lengths as illustrated in FIG. 2. Elongate members 14, 16 interlock over and around lumen 12 to provide complete enclosure of lumen 12 when cap 10 is in a closed position as seen in FIG. 7.

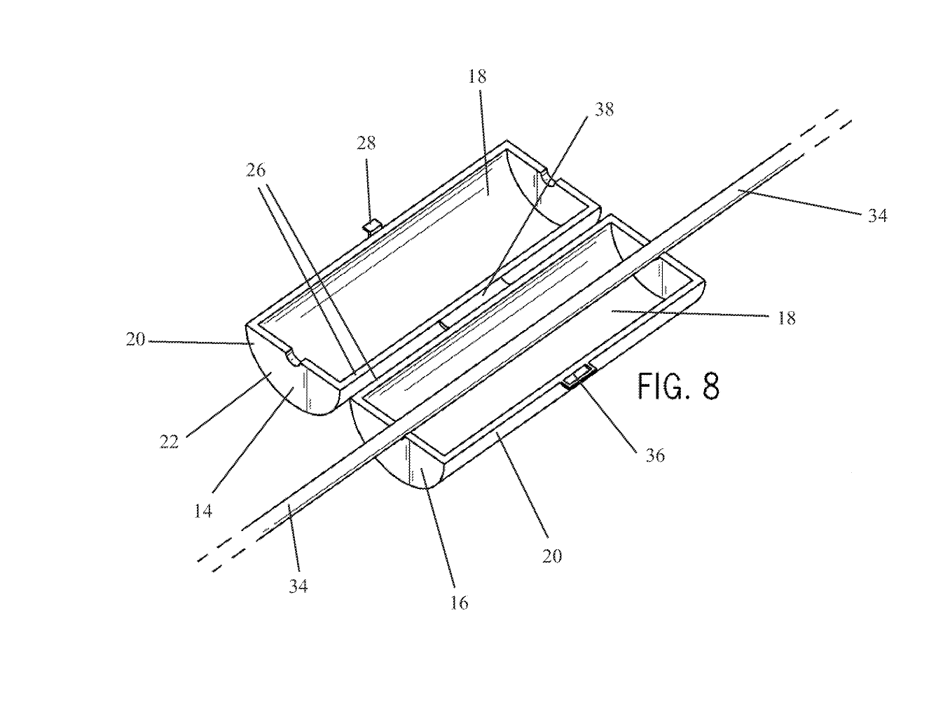

FIGS. 1 and 2 illustrate cap 10 in an open position with lumen 12 placed in contact with cap 10. Proximal and distal ends 22, 24 each include an annular opening 32 for passage of an intravenous line (“IV”) 34 there through as can be seen in FIGS. 1-9, particularly in FIGS. 2 and 8. FIGS. 1-6 show that annular opening 32 on proximal end 22 is identical to annular opening 32 on distal end 24. Annular opening 32 is generally in the range of 1.0 mm to 3.0 mm in diameter, however, annular opening 32 can be any diameter large enough to allow IV line 34 or the like to pass therethrough.

In typical settings, IV line 34 terminates in a lumen 12 as seen in FIGS. 1 and 2. Inside surfaces 18 frictionally engage lumen 12 and prevent removal of lumen 12 when in the closed position as seen in FIG. 7.

Either first or second elongate member 14, 16 includes grip flange 36 for easy opening and closing of cap 10 as illustrated in FIGS. 1-3 and 5. Grip flange 36 can be various sizes and shapes, however it is preferable that grip flange 36 be formed so as to allow easy, one-hand opening and closing of cap 10. First or second elongate member 14, 16 can have one or more grip flanges 36. Grip flange 36 can be integrally formed with either first or second elongate members 14, 16. Grip flange 36 (see FIG. 2) is oriented so that it extends away from pivot axis A. To cap 10, downward thumb pressure is applied on grip flange 36 to pivot first elongate member 14 away from the closed position shown in FIG. 7.

First and second elongate members 14 and 16 of cap 10 may be made of hard plastic materials, a wide choice of which is available and will be apparent to those receiving this disclosure. A wide variety of materials is available for the various parts discussed and illustrated herein.

As seen in FIGS. 1-8, cap 10 includes at least one hinge 38 which connects the two opposite longitudinal edges 26 of first and second elongate members 14, 16. Hinge 38 can be configured similar to a piano hinge or butterfly hinge but is not limited to these hinge configurations.

Cap 10 also includes label-receiving member 40 on the outside surface 20 of first elongate member 14. Label-receiving member 40 can also be on outside sufrace 20 of second elongate member 16. Label-receiving member 40 can be various shapes and sizes as long as label-receiving member 40 is of adequate size for receiving designation of patient care instructions or the like.

Label-receiving member 40 helps to ensure safe labeling of IV lines in a medical setting. Typically in a medical setting, some IV lines are designated for a specific medication or for Total Parenteral Nutrition feeding. Oftentimes, certain medications can only be administered through a particular IV line. For example, an IV line containing a medication such as a “clot buster” could have an adverse effect if the medication itself enters the patient through the wrong IV line. Sometimes a lumen may be designated for use for emergency drugs only and is not to be used for any other purpose. Some lines may become sluggish and are no longer intended for use on a double or triple lumen PICC line but the sluggish/unused lumens remain in the patient.

Cap 10 contains an label-receiving member 40 which is an indicator that can hold patient instructions for a particular lumen/line such as “TPN ONLY,” “DO NOT USE” or “LAB DRAW.” These are just a few examples of the various instructions which could be noted on label-receiving member. The above list is not meant to be exhaustive. Label-receiving member 40 could also be used to write a short message on cap 10 such as “x4321” to contact a pager number/phone number for IV team or the individual that placed a particular IV line.

Cap 10 can also be color-coded (in a wide variety of colors) as set by medical facility policy to designate information such as medications or patient care instructions. For example, a medical facility may designate blue caps for Total Parenteral Nutrition feeding and red caps for lines which are no longer to be used. One skilled in the art will appreciate that there are numerous color-coding designations which can be used.

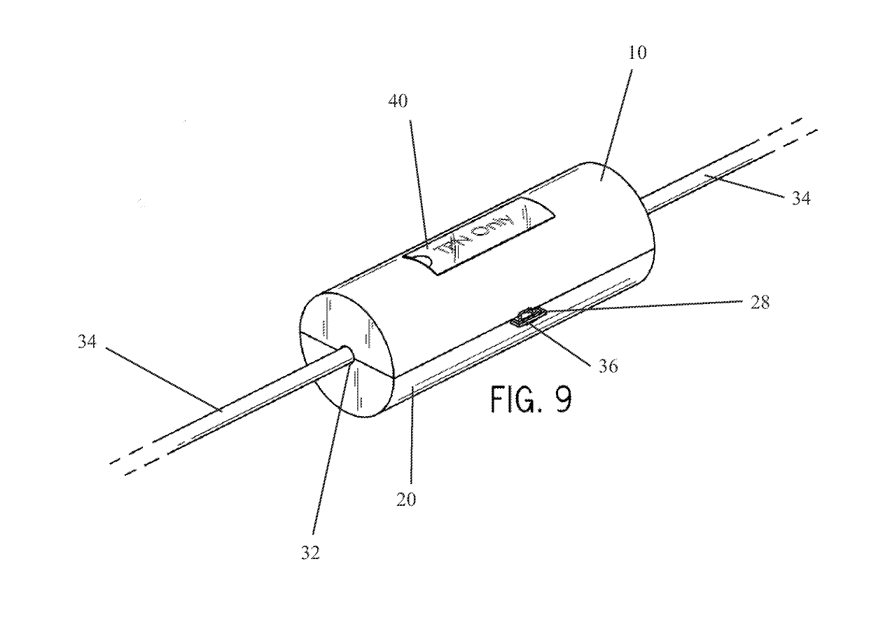

Cap 10 can also be used to cover an IV line 34. When cap 10 is used to cover an IV line, first and second elongate members 14, 16 interlock over and around IV line 34 to provide complete enclosure when cap is in a closed position as seen in FIGS. 8 and 9.

Annular opening 32 is present on both the first and second elongate members 14, 16 and allows IV line 34 to pass completely through cap 10 when in the closed position as seen in FIG. 9. Such a configuration allows cap 10 to be used in either direction and also allows cap 10 to be used on both an IV line 34 and a lumen 12. With this configuration, cap 10 can be moved along an IV line without being completely removed from the line itself in order to label the IV line during use to avoid lumen confusion.

Snap member 28 is designed to temporarily keep cap 10 in a closed position, snap member is not intended to lock onto an IV line 34 or lumen 12. Cap 10 can be easily removed to access lumen 12 or IV line 34.

Cap 10 can cover either an IV line 34 or a lumen 12 as well as the various connectors/devices attached to the lumen 12 or IV line 34. Such connectors/devices may include but are not limited to a threaded-lock cannula, lure-lock cannula, any other connecting end and an IV end cover. Cap 10 encloses one or all of these connectors/devices completely.

First and second elongate members 14 and 16 of cap 10 may be made of hard plastic materials, a wide choice of which is available and will be apparent to those receiving this disclosure. A wide variety of materials is available for the various parts discussed and illustrated herein.

While the principles of this invention have been described in connection with specific embodiments, it should be understood clearly that these descriptions are made only by way of example and are not intended to limit the scope of the invention.

1. An annular cap for covering a lumen comprising:
first and second elongate members each including inside and outside surfaces, proximal and distal end portions and two opposite longitudinal edges; and
an interlocking snap member positioned between and connected to the first and second elongate members along the opposite longitudinal edges, the first and second elongate members being pivotable with respect to each other about a pivot axis,
wherein the elongate members interlock over and around the lumen to provide complete enclosure of the lumen when the cap is in a closed position.

2. The cap of claim 1 wherein the proximal and distal ends each include an annular opening for passage of an intravenous line there through, the intravenous line being connected to the lumen.

3. The cap of claim 2 wherein the annular opening on the proximal end is identical to the annular opening on the distal end.

4. The cap of claim 3 wherein the annular opening is at least 2.0 mm or greater in diameter.

5. The cap of claim 1 wherein the inside surfaces frictionally engage the lumen and prevent removal of the lumen when in the closed position.

6. The cap of claim 1 wherein the second elongate member includes at least one grip flange for opening and closing of the cap.

7. The cap of claim 6 wherein the wherein each grip flange is integrally formed with the second elongate member.

8. The cap of claim 1 further including at least one hinge connecting the two opposite longitudinal edges.

9. The cap of claim 1 further including a label-receiving member on the outside surface of the first elongate member for designation of patient care instructions.

10. The cap of claim 1 further including a label-receiving member on the outside surface of the second elongate member for designation of patient care instructions.

11. The cap of claim 1 wherein the cap is color-coded to designate corresponding medications or patient care instructions.

12. An annular cap for covering an intravenous line comprising:
first and second elongate members each including inside and outside surfaces, proximal and distal end portions and two opposite longitudinal edges; and
an interlocking snap member positioned between and connected to the first and second elongate members along the opposite longitudinal edges, the first and second elongate members being pivotable with respect to each other about a pivot axis,
wherein the elongate members interlock over and around an intravenous line to provide complete enclosure when the cap is in a closed position.

13. The cap of claim 12 wherein the proximal and distal ends each include an annular opening for passage of the intravenous line there through.

14. The cap of claim 13 wherein the annular opening on the proximal end is identical to the annular opening on the distal end.

15. The cap of claim 14 wherein each annular opening is at least 2.0 mm or greater in diameter.

16. The cap of claim 12 wherein each annular opening is sized to allow movement of the cap along a length of the intravenous line.

17. The cap of claim 12 wherein the second elongate member has at least one grip flange positioned for opening and closing of the cap.

18. The cap of claim 12 further including a label-receiving member on the outside surface of the first elongate member for designation of patient care instructions.

19. The cap of claim 12 further including a label-receiving member on the outside surface of the second elongate member for designation of patient care instructions.

20. The cap of claim 12 wherein the cap is color-coded to designate corresponding medications or patient care instructions.
